(19) United States
(12) Patent Application Publication
(10) Pub. No.: US 2014/0173785 A1
(43) Pub. Date: Jun. 19, 2014

(54) Petunia Plant Named 'Archie's Red2Purple'

(71) Applicant: Elmer Lee Archibald, JR., Kernersville, NC (US)

(72) Inventor: Elmer Lee Archibald, JR., Kernersville, NC (US)

(21) Appl. No.: 13/694,640

(22) Filed: Dec. 19, 2012

(51) Int. Cl. A01H 5/02

(52) U.S. Cl. CPC A01H 5/02
USPC 800/323.1

(57) ABSTRACT

A new and distinct flowering Petunia plant with a dark red to purple flower color, vigorous growth habit, and continuous blooming.

### BACKGROUND OF THE INVENTION

The present invention comprises a new and distinct cultivar of petunia plant known by the varietal name ‘Archie's Red2Purple’. The new variety was discovered in April 2012 in a cultivated area as a naturally-occurring sport of many petunia varieties in a greenhouse in North Carolina. The new variety differs from petunia plant ‘Archie's Lime on Yellow’ (concurrently applied-for) in flower color and its vigorous growth habit, however, the new variety is similar to the compared variety in its tolerance to hot weather conditions. The new variety has been found to retain its distinctive characteristics and remain true to type through successive propagations.

The following traits distinguish ‘Archie's Red2Purple’ as a new and distinct cultivar:
1. Dark red top petal color with a purple throat that changes to all purple as the petals age;
2. Upright growth habit that spreads widely;
3. Very vigorous growth habit with abundant branching;
4. Petals exhibit a velvet sheen;
5. Secondary branching of plant is very strong;
6. Variety continuously blooms; and
7. Spent blooms dehydrate and fall from plant.

### DESCRIPTION OF THE DRAWINGS

The accompanying photographic drawings illustrate the new variety, with the color being as nearly true as is possible with color illustrations of this type:

FIG. 1 is a close-up photograph of darkened flowers of the new variety;

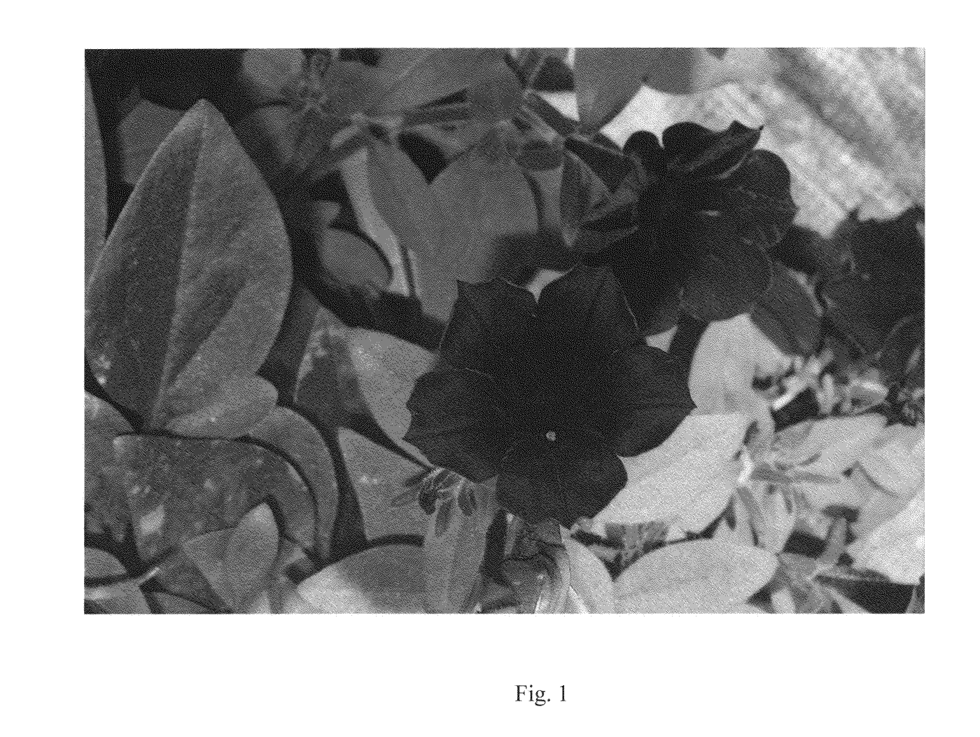

FIG. 2 is a close-up photograph of a younger flower of the new variety;

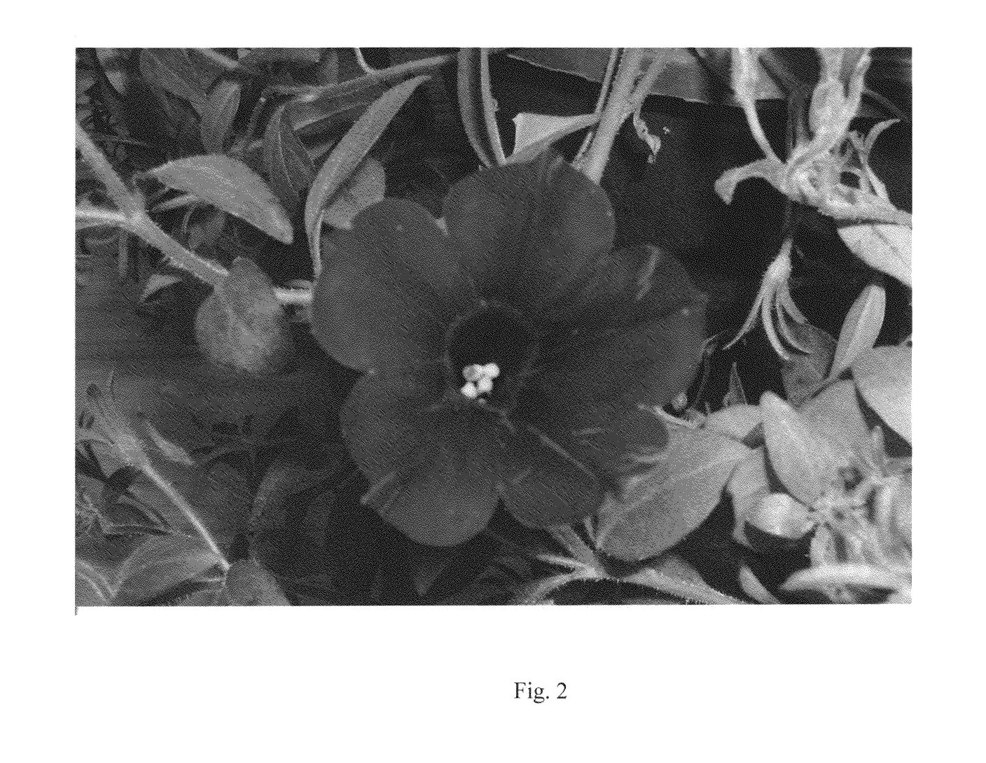

FIG. 3 is photograph of an entire plant of the new variety; and

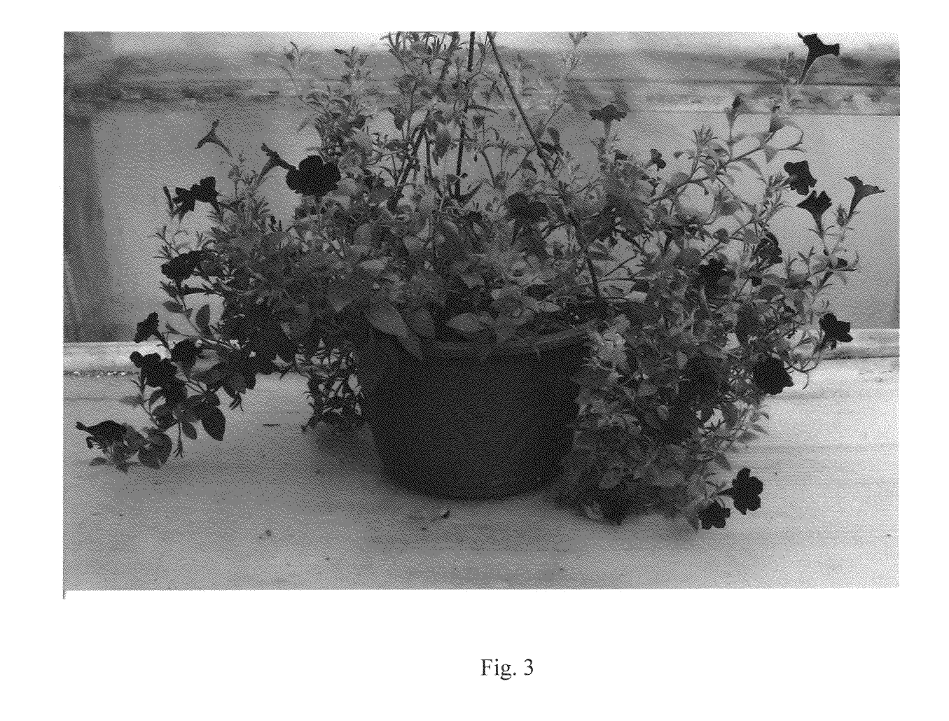

FIG. 4 is a further photograph of an entire plant of the new variety.

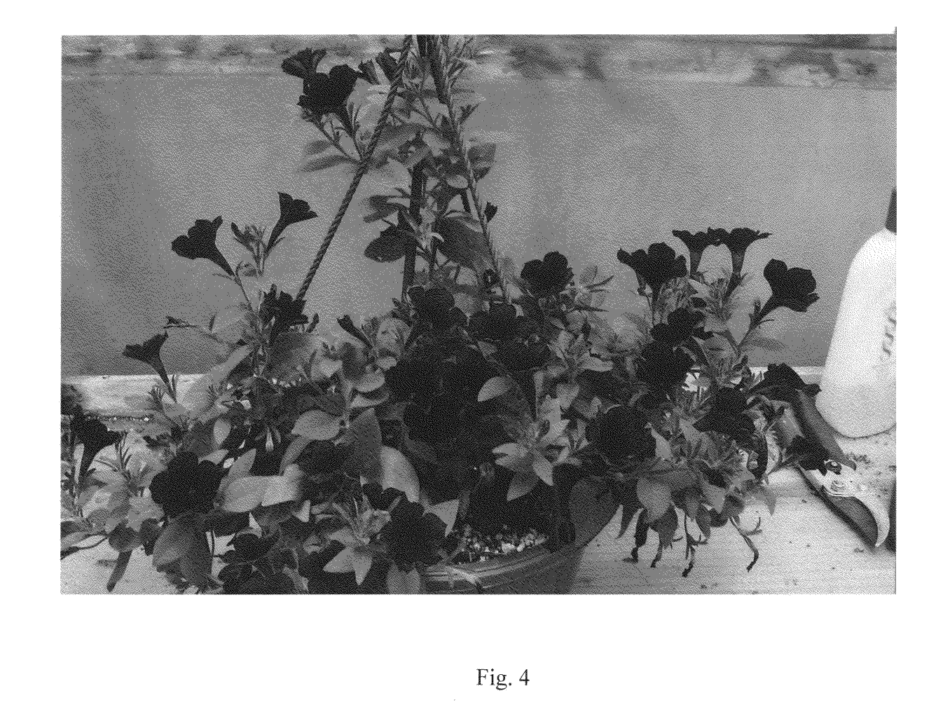

### DESCRIPTION OF THE PLANT

The following detailed description sets forth characteristics of the new cultivar. The data which defines these characteristics were collected by asexual reproductions by tip cuttings carried out in North Carolina in 2012. Plants for the description were 5 months old, grown in a greenhouse under 30% shade cloth in 10 inch hanging baskets. Average day temperatures were 80-90° F. and night temperatures were 50-60° F. The color readings were taken within a covered porch under natural light. Color references are primarily to the 2001 R.H.S. Colour Chart of the Royal Horticultural Society of London.

### Plant

Form: Mounded and spreading.

Height (from soil to top of flowers): 13 inches.

Diameter: 30 inches.

Vigor: High.

Branching habit: High.

Lateral branches:
Form: Angled.
Color: 136B.
Texture: Rough.
Length: 2 inches.
Diameter: 4 inches.
Internode length: ¾ inch.
Quantity: Numerous.

Foliage:
Arrangement: Single.
Number of leaves per branch: Varies by branch length; a 5 inch branch has 30 leaves.
Size of leaf:
   Length: Average of 1 inch.
   Width: From ⅝-1 inch.
Shape of leaf: Ovate.
Shape of apex: Pointed.
Shape of base: Rounded.
Texture: Rough.
Margin: Entire.
Color:
   Young leaves:
      Upper surface: 143A.
      Lower surface: 143B.
   Mature leaves:
      Upper surface: 143A.
      Lower surface: 143C.
Petiole:
   Length: 0.4 cm.
   Diameter: 0.3 cm.
   Color: 143A.
Veins:
   Number: 5.
   Color:
      Upper surface: N79B.
      Lower surface: N79A.

### Flower

Natural flowering season: April through October in North Carolina (Zone 8A).

Flower type and habit: Single; upward; funnel-shaped; 5-petals.

Number of flowers per plant: An average large plant has over 170.

Corolla:
Diameter: 0.4 cm.
Flower tube length: 1.6 cm.
Flower tube diameter at distal end: 0.1 cm.
Flower tube diameter at proximal end: 0.3 cm.
Petal length from throat: 2 cm.
Petal width: 0.8 cm.
Petal quantity: 5 parts fused at base.
Petal apex shape: Circular.
Texture: Velvety.
Color:
   When opening:
      Upper surface: 59A.
      Lower surface: 59B.
   Fully opened:
      Upper surface: N79B.
      Lower surface: N79D.
Flower throat (inside): N79D.
Flower throat, vein: 79.
Flower tube (outside): N79A
Flower tube, vein: 79R
Petal color, fading to: 79A.

Peduncle:
Length: 1.8-1.9 cm.
Diameter: 0.2-0.3 cm.
Color: 143B.
Orientation: Angled.
Strength: Very sturdy.

Bud (described at the beginning of development):
Shape: Trumpet.
Length: 1 cm.
Diameter: 2.5 cm.
Color: N79.

Calyx:
Shape: Cup.
Length: 1.5 cm.
Diameter: 0.1 cm.
Sepal shape: Cup.
Sepal margin: 0.4 cm.
Sepal texture: Rough.
Sepal color:
   Upper surface: 143A.
   Lower surface: 143B.

Disease resistance: No resistance to diseases noted to date.

Fragrance: None.

Weather tolerance: Good tolerance to rain and hot weather.

Lasting quality: Excellent.

Seed/fruit production: None observed.

### Reproductive Organs

Stamens:
Number: 1.
Filament length: 1.6 cm.
Anthers:
   Shape: Capsule.
   Length: 1.2 cm.
   Color: Yellow.
Pollen:
Color: White.
   Amount: Average.

Pistil:
Length: 1.1 cm.
Style:
   Length: 0.9 cm.
   Color: White.
Stigma:
   Shape: Rounded.
   Color: Yellow.
   Ovary color: White.

I claim:

1. A new and distinct variety of Petunia plant substantially as shown and described.
